(19) United States
(12) Patent Application Publication
(10) Pub. No.: US 2026/0048228 A1
(43) Pub. Date: Feb. 19, 2026

(54) VIBRATING HEADBAND

(71) Applicant: Craig Roberts, Halifax, VA (US)

(72) Inventor: Craig Roberts, Halifax, VA (US)

(21) Appl. No.: 18/808,612

(22) Filed: Aug. 19, 2024

(51) Int. Cl. A61M 21/02; A41D 20/00; A61H 23/02; A61M 21/00

(52) U.S. Cl. CPC A61M 21/02; A41D 20/00; A61H 23/02; A41D 2400/322; A61H 2201/501; A61H 2201/5025; A61M 2021/0022; A61M 2209/088; A61M 2210/06

(57) ABSTRACT

A vibrating headband to be worn by a user on a head of the user, the vibrating headband including a main body having a shape to wrap around apportion of the head of the user, an inner lining disposed on an inner surface of the main body, such that the inner lining contacts the portion of the head of the user, a vibrating motor to cause the vibrating headband to vibrate, and a power button to cause the vibrating motor to activate in response to the power button being pressed by the user.

### BACKGROUND

### 1. Field

The present general inventive concept relates generally to a band, and particularly, to a vibrating headband.

### 2. Description of the Related Art

Conventional stress-relief devices are often unavailable or hard to access while individuals are on the go.

Moreover, these conventional stress-relief devices may not offer the level of customization needed to address individual preferences and stress levels.

Therefore, there is a need for a device that provides customizable stress-relief while a user is mobile.

### SUMMARY

The present general inventive concept provides a vibrating headband.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a vibrating headband to be worn by a user on a head of the user, the vibrating headband including a main body having a shape to wrap around apportion of the head of the user, an inner lining disposed on an inner surface of the main body, such that the inner lining contacts the portion of the head of the user, a vibrating motor to cause the vibrating headband to vibrate, and a power button to cause the vibrating motor to activate in response to the power button being pressed by the user.

The main body may further include a central processing unit (CPU) disposed within the main body to communicate with a mobile device, such that the user controls the vibrating motor via the mobile device.

At least one of the mobile device and the power button may control the vibrating motor to change at least one of a user-selected vibration speed, a user-selected vibration intensity, a user-selected vibration pattern, and other user-selected vibration settings

### BRIEF DESCRIPTION OF THE DRAWINGS

These and/or other features and utilities of the present generally inventive concept will become apparent and more readily appreciated from the following description of the embodiments, taken in conjunction with the accompanying drawings ofwhich:

FIG. 1 illustrates a side angled perspective view of a vibrating headband, according to an exemplary embodiment of the present general inventive concept; and

FIG. 2 illustrates a side perspective view of the vibrating headband of FIG. 1 disposed on a head of a user, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,”“adjacent”versus “directly adjacent,”etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Mobile Device 10
Vibrating Headband 100
Main Body 110
Inner Lining 111
Charging Port 112
Power Button 113
Bluetooth Button 114
Battery 115
Central Processing Unit (CPU) 116
Vibrating Motor 117

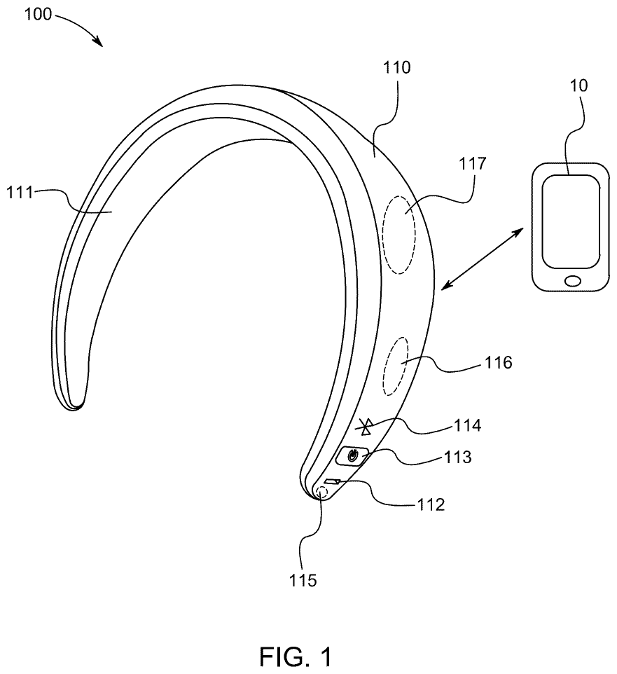

FIG. 1 illustrates a side angled perspective view of a vibrating headband 100, according to an exemplary embodiment of the present general inventive concept.

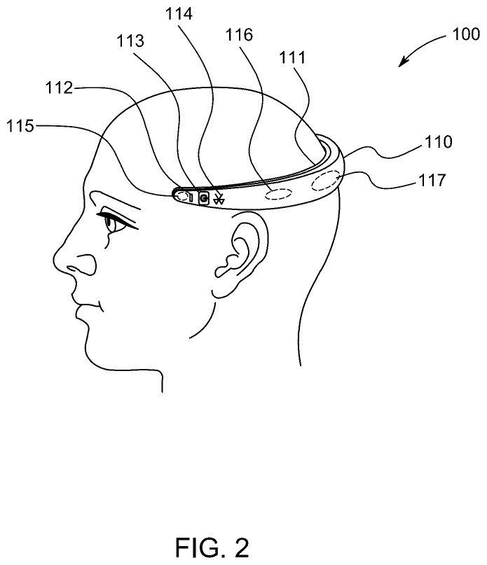

FIG. 2 illustrates a side perspective view of the vibrating headband 100 of FIG. 1 disposed on a head of a user, according to an exemplary embodiment of the present general inventive concept.

The vibrating headband 100, and all components therein and/or connected thereto, may be constructed from at least one of metal, plastic, stone, ceramics, wood, silicone, paper, cloth, circuitry, leather, memory foam, latex, and rubber, etc., but is not limited thereto, and can be constructed from any material known to one of ordinary skill in the art.

Referring to FIGS. 1 and 2, the vibrating headband 100 may include a main body 110, which may include a myriad of components attached thereto and/or disposed thereinside, but is not limited thereto.

The main body 110 may have a shape that is conducive to being wrapped around a back portion of a user's head (e.g., a semicircular shape, a semi-ovular shape, a crescent-moon shape, etc.), such that the vibrating headband 100 may be stably worn by the user on the user's head.

The main body 110 may include an inner lining 111, a charging port 112, a power button 113, a BLUETOOTH button 114, a battery 115, a central processing unit (CPU) 116, and a vibrating motor 117, but is not limited thereto.

The inner lining 111 may be disposed on an inner surface of the main body 110, such that the inner lining 111 directly contacts a head of a user when the user is wearing the vibrating headband 100. Specifically, the inner lining 111 is designed to contact rear and side portions of the user's head, such that the vibrating headband remains comfortably stationary on the user's head.

The inner lining 111 may be constructed from a comfortable memory foam, such that the inner lining 111 customizes itself to the user's head.

The charging port 112, the power button 113, and the BLUETOOTH button 114 may be disposed on an outer surface of the main body 110, such that the user may easily access the charging port 112, the power button 113, and the BLUETOOTH button 114, even if the user is wearing the vibrating headband 100 on the user's head.

The charging port 112 may be designed to receive a connector of a charging cable (not illustrated) therein, in order to charge the battery 115, which may bereplaceable.

The battery 115 may provide power to activate the components within the main body 110, including, but not limited to, the power button 113, the BLUETOOTH button 114, the CPU 116, and the vibrating motor 117.

The power button 113 may be pressed by the user to turn on the vibrating headband 100. In other words, by pressing the power button 113, the battery 115 may supply power to all of the components within the vibrating headband 100, such that the components are ready to perform their respective functions based on the user's activation thereof.

The BLUETOOTH button 114 may allow the vibrating headband 100 to communicate with a mobile device 10 (e.g., via BLUETOOTH) when the BLUETOOTH button 114 is pressed, such that a mobile application running on the mobile device 10 may control vibration speeds, vibration intensities, vibration patterns, and other vibration settings of the vibrating motor 117.

Vibration speeds of the vibrating motor may include slow through quick vibrations. Vibration intensities may include soft through hard vibrations. Vibration patterns may include different types of vibrating massages, such as thumps, rolls, kneads, etc. Other vibration settings may include timers to set a time limit of the vibrations, alarms, etc.

The battery 115, the CPU 116, and the vibrating motor may be disposed within the main body 110.

The battery 115 may be connected to the CPU 116 and the vibrating motor 115 to provide electrical power thereto.

The CPU 116 may include electronic circuitry to carry out instructions of a computer program by performing basic arithmetic, logical, control and input/output (I/O) operations specified by the instructions. The CPU 116 may include an arithmetic logic unit (ALU) that performs arithmetic and logic operations, processor registers that supply operands to the ALU and store the results of ALU operations, and a control unit that fetches instructions from memory and “executes” them by directing the coordinated operations of the ALU, registers and other components. The CPU 116 may also include a microprocessor and a microcontroller. The transmitter CPU 116 may be a local computer device, a remote server, or cloud computing device.

The CPU 116 may also include a storage unit, a random access memory (RAM), a read-only memory (ROM), a hard disk, a flash drive, a database connected to the Internet, cloud-based storage, Internet-based storage, or any other type of storageunit.

The CPU 116 may access the Internet via an internal communication unit to allow the CPU 116 to access a website, and/or may allow for communication with a mobile application and/or a software application. For ease of description, the mobile and/or the software application will be hereinafter referred to as an app. The app may be downloaded from the Internet to be stored on the storage unit of the CPU 116.

The CPU 116 may also have a global positioning service (GPS) device installed therein, to allow the CPU 116 to be located via a GPS signal.

Thus, the CPU 116 may provide communication between the vibrating headband 100 and the mobile device 10, such that the CPU 116 may control the vibrating motor 117 to vibrate at a user-selected vibration speed, a user-selected vibration intensity, a user-selected vibration pattern, and other user-selected vibration settings, based on the user's manipulation of the app on the mobile device 10 that communicates and controls the vibrating headband 100.

Alternatively, the user may press the power button 113 various times to cycles through all of the user-selected vibration speeds, the user-selected vibration intensities, and the user-selected vibration patterns.

In other words, a first press of the power button 113 may electrically connect the battery 115 to the vibrating motor 117 (i.e., provide power to the vibrating headband 100). Any subsequent press of the power button 113 may cause the vibrating motor to cycle through all of the user-selected vibration speeds, the user-selected vibration intensities, and the user-selected vibration patterns. If the user holds down the power button 113, then the vibrating headband 100 may be powered-off.

The present general inventive concept may include a vibrating headband 100 to be worn by a user on a head of the user, the vibrating headband 100 including a main body 100 having a shape to wrap around apportion of the head of the user, an inner lining 111 disposed on an inner surface of the main body 110, such that the inner lining 111 contacts the portion of the head of the user, a vibrating motor 117 to cause the vibrating headband 100 to vibrate, and a power button 113 to cause the vibrating motor 117 to activate in response to the power button 113 being pressed by the user.

The main body 110 may further include a central processing unit (CPU) 116 disposed within the main body 110 to communicate with a mobile device 10, such that the user controls the vibrating motor 117 via the mobile device 10.

At least one of the mobile device 10 and the power button 113 may control the vibrating motor 117 to change at least one of a user-selected vibration speed, a user-selected vibration intensity, a user-selected vibration pattern, and other user-selected vibration settings.

A main purpose of the present general inventive concept is to provide users with a vibrating headband designed to offer soothing and relaxing pulses and vibrations during utilization. Ingenious and practical, the present general inventive concept offers a modern wearable accessory that provides immediate relaxation and stress reduction, helping individuals stay calm and focused throughout the day whether they're commuting, traveling, or working in a busy office environment. Expanding on the initial design of an average headband, the present general inventive concept introduces a novel vibrating, Bluetooth-compatible option that works in complement with a mobile app; thus, allowing for advanced features and seamless integration with a smartphone. Users can easily control the device via their phone and adjust the vibration settings to suit personal stress levels and preferences. Additionally, the accompanying app allows users to download their favorite vibration settings, providing a more trailered experience. Unlike any other traditional stress relief methods that would otherwise require waiting until individuals are home to unwind, the present general inventive concept offers instant relief whether users are traveling on a plane, train, or bus, or simply need a moment of relaxation at their desk due to its compact, wearable yet discreet design. This innovative, top-quality device provides on-the-go stress relief, whenever it's needed; thus, it may prove to be essential in the medical/therapeutic industry.

The present general inventive concept is the only product of its kind that offers a smartphone and Bluetooth-compatible massage accessory able to deliver soothing vibrations directly through a headband. This unprecedented product is uniquely designed with lightweight, breathable materials to provide unparalleled comfort and durability. Its horseshoe design ensures a secure fit, allowing the band to stay in place while the inner lining, made of memory foam materials, enhances comfort. Possessing adjustable vibration settings permits the wearable item to cater to individual preferences, along with its compact design, rechargeable battery, and customizable color schemes and patterns. Ultimately, the present general inventive concept caters to all individuals making it suitable for diverse users, of all ages.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A vibrating headband to be worn by a user on a head of the user, the vibrating headband comprising:
a main body having a shape to wrap around apportion of the head of theuser;
an inner lining disposed on an inner surface of the main body, such that the inner lining contacts the portion of the head of the user;
a vibrating motor to cause the vibrating headband to vibrate; and
a power button to cause the vibrating motor to activate in response to the power button being pressed by the user.

2. The vibrating headband of claim 1, wherein the main body furthercomprises:
a central processing unit (CPU) disposed within the main body to communicate with a mobile device, such that the user controls the vibrating motor via the mobiledevice.

3. The vibrating headband of claim 2, wherein at least one of the mobile device and the power button control the vibrating motor to change at least one of a user-selected vibration speed, a user-selected vibration intensity, a user-selected vibration pattern, and other user-selected vibration settings.
